(19) United States
(12) Patent Application Publication
(10) Pub. No.: US 2019/0015135 A1
(43) Pub. Date: Jan. 17, 2019

(54) CERVICAL PLATE

(71) Applicant: Jill Donaldson, Indianapolis, IN (US)

(72) Inventor: Jill Donaldson, Indianapolis, IN (US)

(21) Appl. No.: 15/648,797

(22) Filed: Jul. 13, 2017

(51) Int. Cl. A61B 17/70; A61B 17/80

(52) U.S. Cl. CPC A61B 17/7059; A61B 17/8061; A61B 17/8033

(57) ABSTRACT

A spinal implant for implantation includes a plate that is adapted to contact the anterior side of an upper and lower vertebral body. An opening defined through the plate is adapted to receive a fastener there through so that the fastener extends into the bone plate of one of the vertebral bodies by extending across a disc space between the vertebral bodies at an oblique angle. An extension portion may extend at least partially in the cranial-caudal direction for an upper or lower edge of the plate to define a recessed portion. The extension portion may be adapted to be positioned adjacent a preexisting spinal device so that the preexisting spinal device is located within the recessed portion.

### BACKGROUND

The present invention relates to spinal implants for implantation on vertebral bodies on the anterior side of the spine.

Spinal implants, such as plates, may be used to stabilize the spine. Often, a plate is attached to a vertebra or vertebrae by screws. Plates are commonly used in the cervical spine, near the patient's neck. Cervical implants may be attached to the anterior or front side of the spine or may be attached from the back or posterior. Already existing spinal implants located on the vertebra or an adjacent vertebra to the desired location of a plate may make it difficult for implantation of the plate. It would be desirable for a spinal implant to be able to be securely attached to the spine while also accommodating for preexisting spinal device positioned near the location where the new spinal implant is needed.

Thus, there is a need for improvement in this field.

### SUMMARY

The claims, and only the claims, recite the invention. In summary, it may include a spinal implant for implantation on the anterior side of an upper vertebral body, including a bottom bone plate, and a lower vertebral body, including a top bone plate. A disc space is defined between the bottom and top bone plates.

The spinal implant includes a plate having an anterior side and a posterior side. The posterior side of the plate is adapted to contact the anterior side of the upper vertebral body and the anterior side of the lower vertebral body.

A first opening is defined through the anterior side and the posterior side of the plate. The first opening is adapted to receive a first fastener there through so that the first fastener extends into one of the vertebral bodies. The first fastener is adapted to extend across the disc space at an oblique angle, and into the bone plate of the one of the vertebral bodies, when said first fastener is inserted through said first opening.

A second opening is defined through the anterior side and the posterior side of the plate. The second opening is adapted to receive a second fastener there through so that said second fastener extends into one of the vertebral bodies.

The plate may also include an upper edge and a lower edge and an extension portion extending at least partially in the cranial-caudal direction from at least one of the edges of the plate to define a recessed portion in said plate. When said plate contacts said upper and lower vertebral bodies, the extension portion may be adapted to be positioned adjacent to a preexisting spinal device located on at least one of the vertebral bodies, so that the preexisting spinal device is positioned within the recessed portion.

In some embodiments, an anterior fastener opening may be defined through the anterior side and the posterior side of the plate. The anterior fastener opening may be adapted to receive an anterior fastener there through so that said anterior fastener extends into one of the vertebral bodies. The anterior fastener may be adapted to extend through the anterior side of one of the vertebral bodies when said anterior fastener is inserted through the anterior fastener opening.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a front view of a spinal implant implanted on the anterior side of an upper and lower vertebral body.

FIG. 2 is a side cross-sectional view of the spinal implant of FIG. 1 attached to an upper and lower vertebral body.

FIG. 3 is a front view of the spinal implant of FIG. 1 including locking members.

FIG. 4A is a front view of an embodiment of a spinal implant.

FIG. 4B is a front view of an embodiment of a spinal implant.

FIG. 4C is a front view of an embodiment of a spinal implant.

FIG. 4D is a front view of an embodiment of a spinal implant.

FIG. 4E is a front view of an embodiment of a spinal implant.

FIG. 4F is a front view of an embodiment of a spinal implant.

FIG. 5 is a side cross-sectional view of a spinal implant attached to an upper and lower vertebral body.

FIG. 6 is a front view of a spinal implant implanted on the anterior side of an upper and lower vertebral body.

FIG. 7 is a side cross-sectional view of the spinal implant of FIG. 6 attached to an upper and lower vertebral body.

### DESCRIPTION OF THE SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

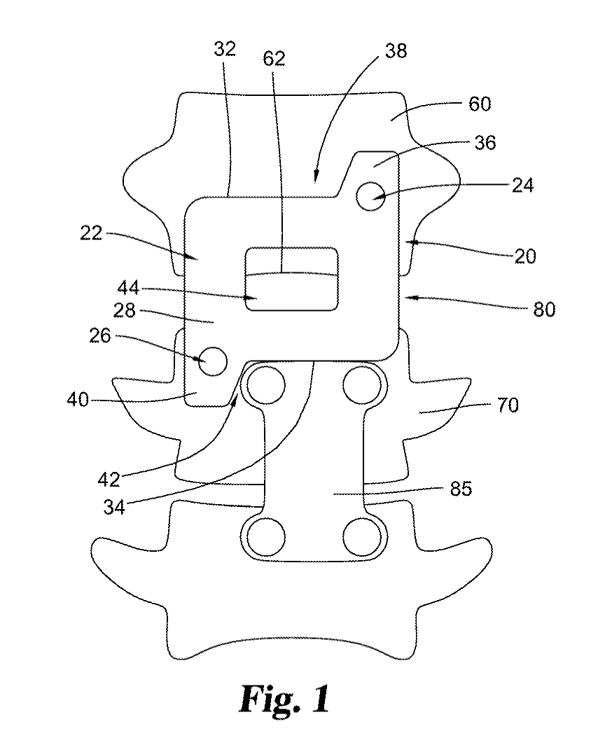

With reference to the drawing Figures, for example FIGS. 1-5, a spinal implant 20 is shown. The spinal implant 20 may be implanted on the anterior side 64 of an upper vertebral body 60 and a lower vertebral body 70. The upper vertebral body 60 includes a bottom bone plate 62 and the lower vertebral body 70 includes a top bone plate 72. A disc space 80 is defined between the bottom and top bone plates 62, 72.

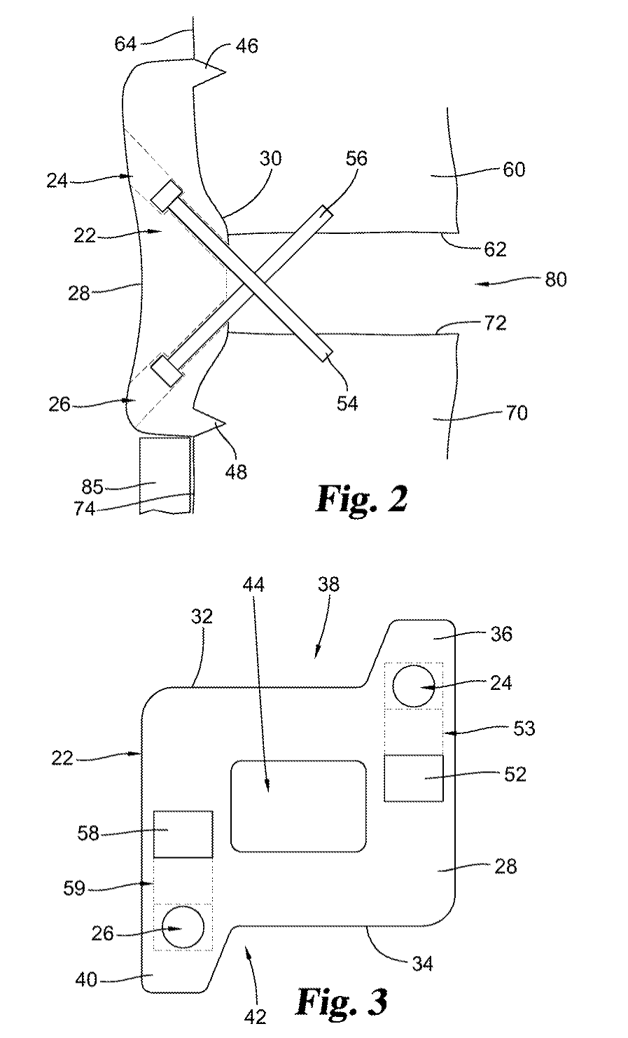

The spinal implant 20 includes a plate 22 having an anterior side 28 and a posterior side 30. The plate 22 may also include an upper edge 32 and a lower edge 34. The posterior side 30 of the plate 22 is adapted to contact the anterior side 64 of the upper vertebral body 60 and the anterior side 74 of the lower vertebral body 70. A first opening 24 is defined through the anterior side 28 and the posterior side 30 of the plate 22. The first opening 24 is adapted to receive a first fastener 54 there through so that the first fastener 54 extends into one of the vertebral bodies. The first fastener 54 is adapted to extend across the disc space 80 at an oblique angle, and into the bone plate 62, 72 of the one of the vertebral bodies 60, 70 when the first fastener 54 is inserted through the first opening 24 (see FIG. 2).

A second opening 26 is defined through the anterior side 28 and the posterior side 30 of the plate 22. The second opening 26 is adapted to receive a second fastener 56 there through so that the second fastener 56 extends into one of the vertebral bodies. In some embodiments, the second fastener 56 is adapted to extend across the disc space 80 at an oblique angle and into the bone plate 62, 72 of one of the vertebral bodies 60, 70 when the second fastener 56 is inserted through the second opening 26.

Inserting a fastener 54, 56 through a bone plate 62, 72 provides several advantages. First, by entering the vertebral body through the bone plate rather than further away from disc space 80, the vertebral bodies are able to maintain more of their normal elasticity despite the presence of spinal implant 20. Also, the fastener acts to compress the end plate of the vertebral body against spinal implant 20 for improved fusion of the implant to the vertebral body.

One feature that is optional, but not required, may be that the first fastener 54 and the second fastener 56 are adapted to extend into separate vertebral bodies. For example, the first fastener 54 may extend into the lower vertebral body 70 while the second fastener 56 may extend into the lower vertebral body 70. Additionally, as another optional feature, the first and second fasteners 54, 56 may be adapted to be countersunk in respective first and second openings 24, 26 so that the fasteners do not extend from the anterior side 28 of the plate 22 when the fasteners are inserted through their respective openings.

One feature that is optional, but not required, may be a tooth 46, 48 that extends posteriorly from the posterior side 30 of the plate 22. The tooth 46, 48 may be adapted to be anchored in one of the vertebral bodies when the spinal implant 20 is in contact with the upper and lower vertebral bodies. Optionally, in some embodiments. the spinal implant 20 may include more than one tooth. One of the teeth 46 may be adapted to be anchored in the upper vertebral body 60 while another tooth 48 may be adapted to be anchored in the lower vertebral body 70.

One feature that is optional, but not required, may be that the posterior side 30 of the plate 22 is made from a porous metal to allow bone growth into the posterior side 30. As an example, in some embodiments, the posterior side 30 of the plate 22 may be made from porous titanium.

One feature that is optional, but not required, may be a locking member 52 located on the anterior side 28 of the plate 22 (see FIG. 3). The locking member 52 may be movable between an open and closed position along a locking member path 53. When the locking member 52 is in the closed position, the first fastener 54 is blocked from withdrawal from the first opening 24. Some embodiments may include locking members for each of the fasteners. For example, an additional locking member 58 may be located on the anterior side 28 of the plate 22. Locking member 58 may be moved along a locking member path 59 to prevent the second fastener 56 from withdrawal from the second opening 26 when locking member 58 is in a closed position.

In other embodiments, other suitable locking mechanisms that prevent withdrawal of a fastener from an opening may be used. For example, the locking member may be a hinged gate that may be moved to cover a fastener, or the locking member may be an expansion ring that may accept and hold a fastener.

One feature that is optional, but not required, may be that the anterior side 28 of the plate 22 is concave. Another optional feature is a window 44 defined through the plate 22 that allows the disc space 80 to be viewed when the spinal implant 20 is in contact with the upper and lower vertebral bodies.

Some embodiments of spinal implant 20 may be adapted for implantation on the anterior side 64, 74 of a vertebral body 60, 70 that includes a preexisting spinal device 85. The spinal implant 20 may include an extension portion 36 or 40 that extends at least partially in the cranial-caudal direction from either the upper edge 32 or the lower edge 34 of the plate 22 to define a recessed portion 38 or 42. When the plate 22 contacts the upper and lower vertebral bodies, extension portion 40 is adapted to be positioned adjacent the preexisting spinal device 85, so that the preexisting spinal device 85 is positioned within the recessed portion 38 or 42. Some embodiments may include only a single extension portion 36, while other embodiments, as shown in FIG. 1, may include multiple extension portions 36, 40. At least a portion of a preexisting spinal device 85 may be positioned within either of the recessed portions 38, 42, or in some embodiments at least a portion of two separate preexisting spinal devices 85 may be positioned in a respective recessed portion 38, 42.

One feature that is optional, but not required, may be that at least two sides of the preexisting spinal device 85 are positioned within the recessed portion 38 or 42. In some embodiments, the extension portion 36 40 and the edge 32, 34 of the plate 22 from which it extends may form an L-shape. The preexisting spinal device 85 may be positioned within the recessed portion formed by the L-shape so that the extension portion 36, 40 and either the upper edge 32 or the lower edge 34 borders the preexisting spinal device 85 on at least two sides.

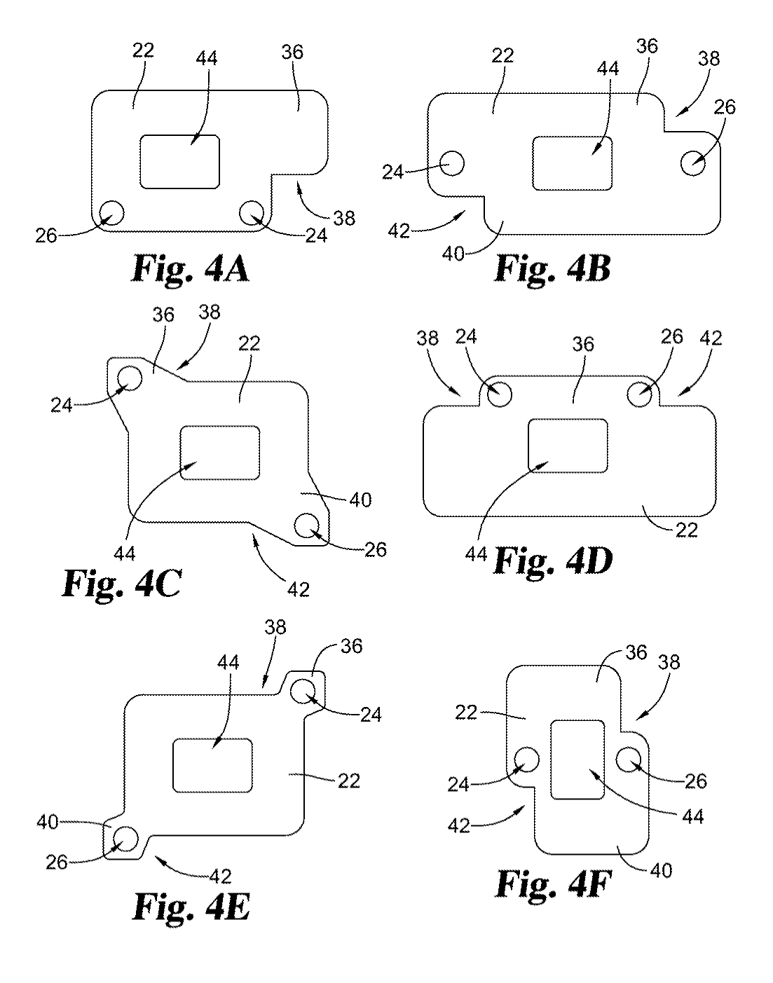

FIGS. 4A-4F illustrate additional embodiments of spinal implant 20. As shown, in other embodiments, plate 22 may have different shapes to accommodate different preexisting spinal devices 85. The extension portions 36, 40 extending from plate 22 may have varying shapes or positions on plate 22 as desired. Additionally, openings 24, 26 may be positioned at different places on plate 22 or on extension portions 36, 40.

Although FIG. 1 shows spinal implant 20 positioned above a preexisting spinal device 85, it is not required that spinal implant 20 is used in conjunction with the preexisting spinal device 85. In some embodiments, spinal implant 20 may be implanted on vertebral bodies 60, 70 during an initial procedure without a preexisting spinal device 85.

As shown in FIG. 5, in some embodiments, the angle at which the openings for first fastener 54 and second fastener 56 extend through spinal implant 20 may be varied. In FIG. 5, first opening 124 and second opening 126 are positioned at steeper angles than first opening 24 and second opening 26 (see FIG. 2) so that fasteners 54, 56 enter bone plates 62, 72 at a more anterior position than in the embodiment shown in FIG. 2.

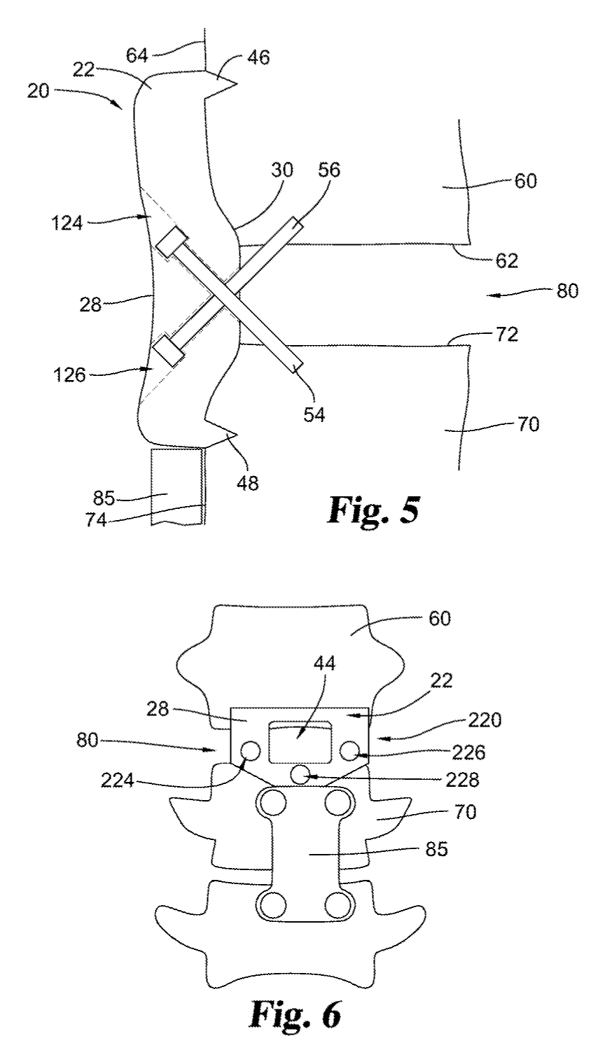

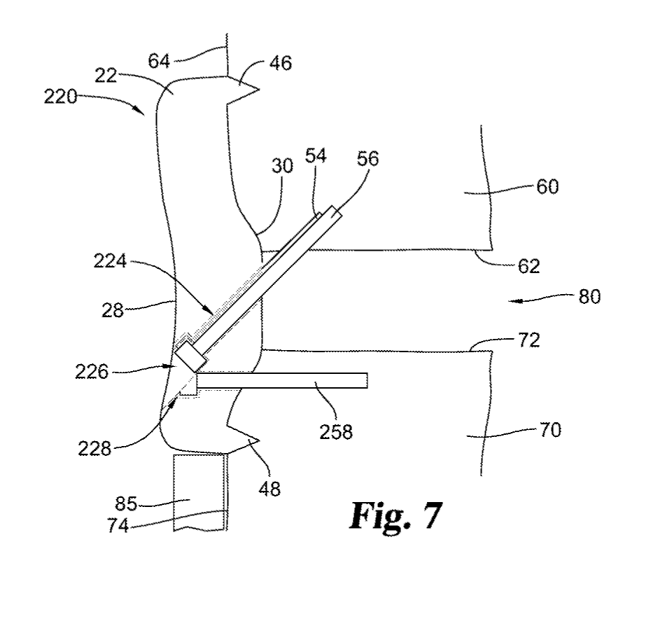

With reference to FIGS. 6-7, an alternative embodiment of a spinal implant 220 is shown. The spinal implant 220 includes a first opening 224 and a second opening 226 defined through plate 22. The first opening 224 is adapted to receive a first fastener 54 there through so that the first fastener 54 extends into a vertebral body 60, 70. The second opening 226 is adapted to receive a second fastener 56 there through so that the second fastener 56 also extends into the same vertebral body 60, 70 as first fastener 54. The spinal implant 220 may include an anterior fastener opening 228 defined through the plate 22 that is adapted to receive an anterior fastener 258 there through so that the anterior fastener 258 extends into the anterior side 64, 74 of one of the vertebral bodies 60, 70.

In the embodiment shown, both the first fastener 54 (shown behind second fastener 56 in FIG. 7) and second fastener 56 extend across disc space 80 and through bone plate 62 into upper vertebral body 60. Anterior fastener 258 extends into lower vertebral body 70 through anterior side 74 without crossing through disc space 80. In other embodiments, the first fastener 54 and second fastener 56 may extend across disc space 80 and through bone plate 72 into lower vertebral body 70 while anterior fastener 258 extends into upper vertebral body 60.

As used here (claims, specification, and other definitions) the following terms have the following meaning:

Articles and phases such as, “the”, “a”, “an”, “at least one”, and “a first”, “comprising”, “having” and “including” here are not limited to mean only one, but rather are inclusive and open ended to also include, optionally, two or more of such elements and/or other elements. In terms of the meaning of words or terms or phrases herein, literal differences therein are not superfluous and have different meaning, and are not to be synonymous with words or terms or phrases in the same or other claims.

The term “means for” in a claim invokes 35 U.S.C. § 112(f), literally encompassing the recited function and corresponding structure and equivalents thereto. Its absence does not, unless there otherwise is insufficient structure recited for that claim element. Nothing herein or elsewhere restricts the doctrine of equivalents available to the patentee.

The term “and/or” is inclusive here, meaning “and” as well as “or”. For example, “P and/or Q” encompasses, P, Q, and P with Q; and, such “P and/or Q” may include other elements as well.

The term “adjacent” as used herein has the meaning, being next to or proximate another object. Two objects may be adjacent if they are in contact with each other or if they are merely in proximity to each other without actual contact.

The term “anterior” as used herein has the meaning, being situated nearest or toward the front of the body. With respect to the spine, the anterior is considered to be the side of the spine closest to the stomach or the throat.

The term “bone plate” as used herein has the meaning, the layer of thickened bone that is found at the top or bottom portion of the vertebral body. The bone plate is the portion of a vertebra that interfaces with the vertebral disc between vertebrae.

The term “border” as used herein has the meaning, to be positioned beside or along an edge of an object. One object may border another object by either contacting the object or by being near the object without contact. Additionally, an object may border another object on one side by being positioned next to the entire length of that side or by being positioned next to only a portion of the entire length of that side.

The term “cranial-caudal direction” as used herein has the meaning, in the direction along an axis running vertically toward and away from a (standing) patient's skull.

The term “closed position” as used herein has the meaning, a location for a locking member in which the locking member prevents removal of a fastener from an opening.

The term “concave” as used herein has the meaning, having a surface that curves inward. With respect to an object located on the anterior side of the spine, a concave surface is curved in the direction of the spine.

The term “countersunk” as used herein has the meaning, inserting a fastener into an object so that the fastener is flush with or below the surface of the object in which the fastener has been inserted.

The term “disc space” as used herein has the meaning, the cavity between adjacent vertebrae in which the vertebral disc normally resides.

The term “extension portion” as used herein has the meaning, a part of the spinal implant that extends an increased length from a portion of an edge of the plate. The extension portion may be on any edge of the plate. A plate may include a single extension portion or may include multiple extension portions from the same edge of the plate or extension portions on multiple edges of the plate. In some embodiments, the extension portion may extend from an edge of the plate at a right angle; however, in other embodiments the extension portion may extend at an oblique angle.

The term “fastener” as used herein has the meaning, an object used to attach and secure one object to another object. A fastener may be an anchor, a bolt, a screw, or any other suitable object used for attachment. The fastener may have a threaded or serrated end or may have a smooth end. In some embodiments, the fastener may be a lag screw.

The term “L-shape” as used herein has the meaning, a shape that is generally in the form of the capital letter “L.” formed by a longer side connected to a shorter side. The sides may be connected at a right angle or at an angle that is close to or approximately a right angle. The angle between the two sides may also be curved. An L-shaped object may be oriented in the same direction as a capital letter “L” or may be rotated.

The term “locking member” as used herein has the meaning, an object used to prevent the movement of another object or objects. For example. a locking member may be any structure used to prevent the movement of a fastener. A locking member may be fixed or may be movable. The locking member may be hinged, may slide, or may use any other suitable type of motion to allow the locking member to prevent movement of a fastener. In some embodiments, the locking member may include expansion rings that expand to accept and hold a fastener.

The term “lower edge” as used herein has the meaning, the lower limit of an object where lower refers to the direction toward a person's feet when the object is located on the body.

The term “oblique” as used herein has the meaning, angled so as to be neither perpendicular to nor parallel to a specified axis.

The term “open position” as used herein has the meaning, a location for a locking member in which the locking member allows removal of a fastener from an opening.

The term “opening” as used herein has the meaning, an aperture or a gap extending through an object. An opening may extend entirely through the object. Some openings may allow access for another object to be inserted.

The term “plate” as used herein has the meaning, the body of or the main or principal part of the spinal implant. For example. the plate is the portion of the spinal implant that is positioned on the anterior side of the vertebrae.

The term “posterior” as used herein has the meaning, being situated nearest or toward the back of the body. With respect to the spine, the posterior is considered to be the side of the spine closest to the patient's back. The posterior is the opposite side from the anterior.

The term “posteriorly” as used herein has the meaning, positioned in a location more posterior with respect to another object where the posterior refers to a position nearer to the back of a reference point.

The term “porous metal” as used herein has the meaning, a metal structure that includes spaces, holes, or openings that allow passage of gases, liquids, or solids. The size of the holes may be uniform or may be of varying sizes as desired.

The term “preexisting spinal device” as used herein has the meaning, a device that is already present on or within the spine prior to attachment of the spinal implant. As an example, a preexisting spinal device may be a plate, a cage, a rod, a screw, or any other device that may be attached to the spine.

The term “recessed portion” as used herein has the meaning, the open space created between the edge of a plate and the extension portion extending from the edge. One side of the recessed portion is defined by the extension portion and another side of the recessed portion is defined by the edge of the plate.

The term “spinal implant” as used herein has the meaning, a device attached to or positioned on the spine by a medical procedure. A spinal implant may be used to stabilize or strengthen the spine, or may be used to correct a deformity. Some examples of spinal implants may include, but are not limited to, rods, pedicle screws, hooks, plates, or cages.

The term “tooth” as used herein has the meaning, a portion of the posterior side of a plate that is able to be at least partially inserted into a vertebral body to assist to hold the plate to a vertebra or vertebrae. A tooth may extend from the posterior side of the plate and may be sharpened or serrated to assist with securement to the vertebral body. A tooth may be secured to the vertebral body by applying force to the plate, for example, manually or through the use of a tool like a hammer.

The term “upper edge” as used herein has the meaning, the upper limit of an object where upper refers to the direction toward a person's head when the object is located on the body.

The term “vertebral body” as used herein has the meaning, the largest, cylindrical part of a vertebra. The vertebra is a bone or object that forms part of the spinal column. The vertebral body could be composed of bone or it can be an implant formed from any variety of materials that would be useful to replace bone such as titanium, stainless steel, or other metallic or non-metallic compounds.

The term “window” as used herein has the meaning, a portion of the plate that allows the disc space to be viewed. For example, a window may be an opening that extends through the plate, or a window may be a portion of the plate that is made from a transparent material through which the disc space is visible.

The invention may include any one or more articles or devices made by any of the claimed methods and/or may by different methods but with a claimed composition.

The language used in the claims and the written description and in the above definitions is to only have its plain and ordinary meaning, except for terms explicitly defined above. Such plain and ordinary meaning is defined here as inclusive of all consistent dictionary definitions from the most recently published (on the filing date of this document) general purpose Webster's dictionaries and Random House dictionaries. While the invention has been illustrated and described in detail in the drawings and foregoing description, the same is to be considered as illustrative and not restrictive in character, it being understood that only the preferred embodiment has been shown and described and that all changes, equivalents, and modifications that come within the spirit of the inventions defined by following claims are desired to be protected. All publications, patents, and patent applications cited in this specification are herein incorporated by reference as if each individual publication, patent, or patent application were specifically and individually indicated to be incorporated by reference and set forth in its entirety herein.

1. A spinal implant for implantation on an anterior side of an upper vertebral body, the upper vertebral body including a bottom bone plate, and a lower vertebral body, the lower vertebral body including a top bone plate, and wherein a disc space is defined between the bottom and top bone plates, the spinal implant comprising:
a plate having an anterior side and a posterior side, wherein said posterior side of said plate is adapted to contact the anterior side of the upper vertebral body and the anterior side of the lower vertebral body;
a first opening defined through said anterior side and said posterior side of said plate adapted to receive a first fastener there through so that said first fastener extends into one of the vertebral bodies;
a second opening defined through said anterior side and said posterior side of said plate adapted to receive a second fastener there through so that said second fastener extends into one of the vertebral bodies; and,
wherein said first fastener is adapted to extend across the disc space at an oblique angle, and into the bone plate of the one of the vertebral bodies when said first fastener is inserted through said first opening.

2. The spinal implant of claim 1, wherein the posterior side of the plate is made from a porous metal to allow bone growth into the posterior side.

3. The spinal implant of claim 2, wherein said posterior side is made from porous titanium.

4. The spinal implant of claim further comprising,
a tooth extending posteriorly from said posterior side of said plate; and,
wherein said tooth is adapted to be anchored in one of the vertebral bodies when the spinal implant is in contact with the upper and lower vertebral bodies.

5. The spinal implant of claim 1, wherein said second fastener is adapted to extend across the disc space at an oblique angle, and into the bone plate of the one of the vertebral bodies when said second fastener is inserted through said second opening.

6. The spinal implant of claim 5, further comprising:
an anterior fastener opening defined through said anterior side and said posterior side of said plate adapted to receive an anterior fastener there through so that said anterior fastener extends into one of the vertebral bodies; and,
wherein said anterior fastener is adapted to enter the anterior side of the vertebral body.

7. The spinal implant of claim 5, wherein said first fastener and said second fastener are adapted to extend into separate vertebral bodies.

8. The spinal implant of claim 1, wherein said first and second fasteners are adapted to be countersunk in said respective first and second openings so that said fasteners do not extend from the anterior side of said plate when said fasteners are inserted through said openings.

9. The spinal implant of claim 1, further comprising:
a locking member located on said anterior side of said plate, said locking member movable between an open and closed position: and,
wherein when said locking member is in said closed position, said first fastener is blocked from withdrawal from said first opening.

10. The spinal implant of claim 1, wherein the anterior side of said plate is concave.

11. The spinal implant of claim 1, further comprising a window defined through said plate that allows the disc space to be viewed when the spinal implant is in contact with the upper and lower vertebral bodies.

12. A spinal implant for implantation on an anterior side of an upper vertebral body and a lower vertebral body, wherein the anterior side of at least one of said vertebral bodies includes a preexisting spinal device, the spinal implant comprising:
a plate having an upper edge, a lower edge, an anterior side, and a posterior side, wherein said posterior side of said plate is adapted to contact the anterior side of the upper vertebral body and the anterior side of the lower vertebral body;
an extension portion extending at least partially in a cranial-caudal direction from at least one of said upper edge or said lower edge of said plate to define a recessed portion in said plate; and,
wherein when said plate contacts said upper and lower vertebral bodies, the extension portion is adapted to be positioned adjacent the preexisting spinal device, so that the preexisting spinal device is positioned within the recessed portion.

13. The spinal implant of claim 12, further comprising:
a first opening defined through said anterior side and said posterior side of said plate adapted to receive a first fastener there through so that said first fastener extends into one of the vertebral bodies.

14. The spinal implant of claim 13,
wherein the upper vertebral body includes a bottom bone plate, the lower vertebral body includes an top bone plate, and wherein a disc space is defined between the bottom and top bone plates; and,
wherein said first fastener is adapted to extend across the disc space at an oblique angle, and into the bone plate of the one of the vertebral bodies when said first fastener is inserted through said first opening.

15. The spinal implant of claim 13, further comprising:
a second opening defined through said anterior side and said posterior side of said plate adapted to receive a second fastener there through so that said second fastener extends into one of the vertebral bodies.

16. The spinal implant of claim 15,
wherein the upper vertebral body includes a bottom bone plate, the lower vertebral body includes an top bone plate, and wherein a disc space is defined between the bottom and top bone plates;
wherein said first fastener is adapted to extend across the disc space at an oblique angle, and into the bone plate of the one of the vertebral bodies when said first fastener is inserted through said first opening; and,
wherein said second fastener is adapted to extend across the disc space at an oblique angle, and into the bone plate of the one of the vertebral bodies when said second fastener is inserted through said second opening.

17. The spinal implant of claim 15, further comprising:
a locking member located on said anterior side of said plate, said locking member movable between an open and closed position; and,
wherein when said locking member is in said closed position, said first and second fasteners are blocked from withdrawal from said respective first and second openings.

18. The spinal implant of claim 12, wherein the posterior side of said plate is made from a porous metal to allow bone growth into the posterior side.

19. The spinal implant of claim 12, further comprising:
a tooth extending posteriorly from said posterior side of said plate; and,
wherein said tooth is adapted to be anchored in one of the vertebral bodies when the spinal implant is in contact with the upper and lower vertebral bodies.

20. The spinal implant of claim 12, wherein the anterior side of said plate is concave.

21. The spinal implant of claim 12, wherein at least two sides of the preexisting spinal device are positioned within the recessed portion.

22. The spinal implant of claim 12,
wherein said extension portion and either said upper edge or said lower edge from which it extends forms an L-shape; and,
wherein said extension portion and either said upper edge or said lower edge borders the preexisting spinal device on at least two sides.

23. A spinal implant for implantation on an anterior side of an upper vertebral body, the upper vertebral body including a bottom bone plate, and a lower vertebral body, the lower vertebral body including a top bone plate, and wherein a disc space is defined between the bottom and top bone plates, the spinal implant comprising:
a plate having an anterior side and a posterior side, wherein said posterior side of said plate is adapted to contact the anterior side of the upper vertebral body and the anterior side of the lower vertebral body;
a first opening defined through said anterior side and said posterior side of said plate adapted to receive a first fastener there through so that said first fastener extends into one of the vertebral bodies;
an anterior fastener opening defined through said anterior side and said posterior side of said plate adapted to receive an anterior fastener there through so that said anterior fastener extends into one of the vertebral bodies;
wherein said first fastener is adapted to extend across the disc space at an oblique angle, and into the bone plate of the one of the vertebral bodies when said first fastener is inserted through said first opening; and,
wherein said anterior fastener is adapted to extend through the anterior side of one of the vertebral bodies when said anterior fastener is inserted through said anterior fastener opening.
